(19) United States
(12) Patent Application Publication
(10) Pub. No.: US 2019/0183694 A1
(43) Pub. Date: Jun. 20, 2019

(54) Protective cover for frontal genital region

(71) Applicant: Peter O. Owino, Plainsboro, NJ (US)

(72) Inventor: Peter O. Owino, Plainsboro, NJ (US)

(21) Appl. No.: 15/846,138

(22) Filed: Dec. 18, 2017

(51) Int. Cl. A61F 13/84; A61F 13/491

(52) U.S. Cl. CPC A61F 13/84; A61F 5/451; A61F 13/491

(57) ABSTRACT

A genital protective cover containing a suitable fluid penetrable mask locatable over the genital region to prevent the exposure of the genital to fecal matter collected in and migrating from the neighboring buttock region, wherein the protective cover is secured using the suitable material to keep the protective cover stick to the body on and/or around the genital region.

### CROSS-REFERENCE TO RELATED APPLICATIONS

This application claims priority under 35 U.S.C. § 119 of U.S. Provisional Application Ser. No. 62/436,063 filed on 19 Dec. 2016.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable

### STATEMENT REGARDING PRIOR DISCLOSURES BY THE INVENTOR OR A JOINT INVENTOR

### Field of the Invention

The present invention is directed generally to the undergarments and specifically to genital protective cover used to mask human genitalia to prevent them to be exposed to the fecal matter collected in diaper or any garment worn over it.

### Background of Invention

Parenting is a demanding job which includes countless cumbersome tasks. One major task related to the parenting is to manage baby's bowel movement. A baby should have at least two to five bowel movements in a twenty-four hour period. This might have made Mrs. Valerie Hunter Gordon to invent the first disposable diaper in 1948. Since then, there have been so many changes in its design for removing the difficulties in its existing art and making use of it for patients and elderly persons. It makes disposable diapers become a staple across the world. The diapers are used to absorb and retain waste fluid and collect and contain fecal matter of its user. It helps the user protect the clothing worn and the place used by the wearer from getting soiled and stained. The disposable diapers also have found large markets with the patients and elderly and infirm people.

A difficulty presently associated with the current art is the movement of the fecal matter to genital area. Since the storage area of the fecal matter situates at the bottom of the diaper at or near the perineum, the fecal matter may be prone to flowing or being forced by the overlying pressure to the front part of the user's body and coming into contact with the user's genitalia.

The user's stool mixed with their urine takes form of paste that may freely permeate the entire body in the diaper or clothing worn by the users. It poses threat of serious infections to the user's genitalia and urinary meatus. The users taking antibiotics, or breastfed babies whose mothers are on antibiotics, sometimes get yeast infections because these drugs kill, in addition to the harmful bacteria, the healthy bacteria that keep yeast in check.

The surgeries such as paediatric circumcision, hypospadias surgery or genital infections such as genital herpes result in the exposure of a sensitive meatus, infected genitalia, skin or surgical site to the human stool which may have crossed perineum within the diaper or clothing. The stool houses variety of bacteria and pH concentrations that are injurious to the skin. It may cause host of severe postoperative complications.

Some inventors tried to tackle this issue in the past too. For example, an art registered under Pat. No. US20070197983 provides a genital shield with a rigid, fluid penetrable body located over the genital region to keep the surface of the diaper away from contacting the genitals. It may be useful in limited medical conditions, but it doesn't address the needs of babies and adults for a long term use. Firstly, the rigid edges of the shield exert pressure on the arteries supplying blood to the genital. However, if it was made of flexible material, then it might have failed to withstand the overlying pressure of the diaper and may not serve the purpose it was made for. Secondly, it allows the urine stored in the shield before it passes the permeable wall of the shield as the urine flows from urinary meatus faster than it leaves the permeable shield wall. Thirdly, it is specially designed for patients who have gone through the surgeries. Such patients are normally advised to remain immovable for most of the time. The foregoing points render the past art not suitable for babies and adults.

In addition, a scrotal catch registered under U.S. Pat. No. 8,142,407 B2 is a garment with a scrotal catch. The scrotal catch is attached to the garment separating the interior surface of the garment from an anterior compartment. It supports, protects, and/or covers a male's scrotum in addition to reducing and/or preventing fecal matter from entering the anterior compartment. This art addresses the needs of male patients only. The scrotal catch or cup is made of a non-flexible material which also puts pressure around the male genital. This art intends to cover portion of male's scrotum only. The cup of the scrotal catch does also store the urine before it exits the cup walls similar to the genital shield discussed above.

The prior arts have failed to address the problem of exposure of the fecal matter to the genitals generally in all situations.

### BRIEF SUMMARY OF THE INVENTION

The present invention is a genital protective cover with different shapes for male and female wearers, which is worn beneath a diaper, incontinence wear or undergarment to mask the genital area to protect it from fecal matter migrating from the buttock area. The genital protective cover has one belt-like loop and two pairs of straps. The belt-like loop encircles the waist and is fastened at the back with the help of any suitable method of fastening. The protective cover is further secured against the user's body from lower side using two pair of straps; first pair of straps, while carrying both sides of the mask, runs from perineal region under the genital area making hard v-shape and is attached to the waist loop from the front side; and the second pair originates from the perineal region encircling the buttocks from bottom and both of their sides and joins waist loop where it is attachable to the waist loop. The upper side of the mask is attached to the waist loop. Being adherent to the areas around the genitalia, the mask completely covers the genitalia from all sides. The genital protective cover leaves the buttock area uncovered so that the wearer can pass fecal matter into a diaper, undergarment, or toilet.

The mask is made of a material that allows urine and other discharges from the human genitalia to pass through the walls of the mask but prevents the fluids and fecal matter to flow back through the same so that the wearer's genitalia is not exposed to the fluids and fecal matter. Given the sensitive nature of genitalia, the material forming the mask wall is intended to be soft and flexible to protect it from pressure and abrasion.

The mask may be multilayered with one layer made of an absorbent material inserted between the two non-woven material layers. The genital protective cover has variety of sizes and designs as it is intended to be used by all age groups ranging from infants to elderly people or people with medical conditions or the patients who have gone through surgeries relating to genitalia.

### BRIEF DESCRIPTION OF THE DRAWINGS

FIG. 1 shows an infant wearing a genital protective cover;

FIG. 2A shows a front view of the genital protective cover;

FIG. 2B shows a front view of the genital protective cover with open straps;

FIG. 3A is a side view of the genital protective cover with double layer mask;

FIG. 3B is a side view of the genital protective cover with a four layered mask;

FIG. 4A is a side view of the genital protective cover for a male;

FIG. 4B is a side of a genital protective cover for a male with open straps;

FIG. 5A is a front view of a female genital protective cover;

FIG. 5B is a front view of a female genital protective cover with open straps;

FIG. 6 is a back view of the genital protective cover worn by an infant.

FIG. 7 shows a female genital protective cover worn by an infant;

FIG. 8A shows a front view of a female genital protective cover;

FIG. 8B shows a front view of a female genital protective cover with open straps;

FIG. 9A shows a side view of a female genital protective cover;

FIG. 9B shows a side view of a female protective cover with open straps;

FIG. 10A shows a view of a female genital protective cover;

FIG. 10B shows a view of a female genital protective cover with open straps;

FIG. 11 shows a drawing in which the principle of genital protective cover is introduced to shorts-like garment; and

FIG. 12 is a diaper, napkin or other clothing worn over the genital protective cover.

### DETAILED DESCRIPTION OF THE INVENTION

Within the context of this specification, following terms and expressions will include the following meaning.

“Absorbent pad” refers to the layer of the four layered mask which is located immediately next before the interior layer and is assigned reference number 4b.

“Anterior straps” refer to the straps with reference number 8 that are attached to the left and right sides of the mask and are originated from the bottom of the protective cover and join the waist loop from the front.

“Barrier ply” refers to the layer forming part of the four layered mask and is located next to the exterior layer and is given reference number 4c.

“Exterior layer” refers to the first layer of the multilayer mask with reference number 4d.

“Genital protective cover” refers to the current invention.

“Interior layer” means the last layer of the multilayer mask with reference number 4a which comes in contact with the genital.

“Perineal region” refers to the region between human genitalia and anus.

“Posterior straps” refers to the straps with reference 12 rising from the bottom of the protective cover, enclosing the buttocks and joining the waist loop from the sides of the genital protective wear.

“Mask” refers to the mask protecting the genitalia from fecal matter and is assigned reference number 4.

“Posterior straps ends” mean the ends with reference number 14a and 14b which are attachable to the waist loop 6 on both sides.

“Straps junction” refers to the part of the genital protective cover located at the bottom of the mask in the perineal region and is given reference number 10.

“Shorts-like garments” refers to a garments based on the principles of current invention resembling to the shorts with the posterior part absent exposing the wearer's buttocks and is assigned reference 20.

“Waist loop” means the belt-like loop with reference number 6 to which the anterior and posterior straps and the mask are attached.

“Waist loop ends” refer to the place with reference number 18 where both ends of the waist loop are fastened.

The present invention is directed to a protective cover which is used to protect human genital from exposure to fecal matter collected in the diaper or any garment. The present invention can be used with any suitable garment or absorbent article which may cover the area where anus and genital situate. Such suitable garments or absorbent articles may include, without limitation, diapers, undergarments, training pants, incontinence products, pants, shorts or any other garments. The present invention may be used for babies, elderly and infirm persons and patients who may have to defecate in the diaper or any garments worn by them due to their tender age or infirmity or medical condition.

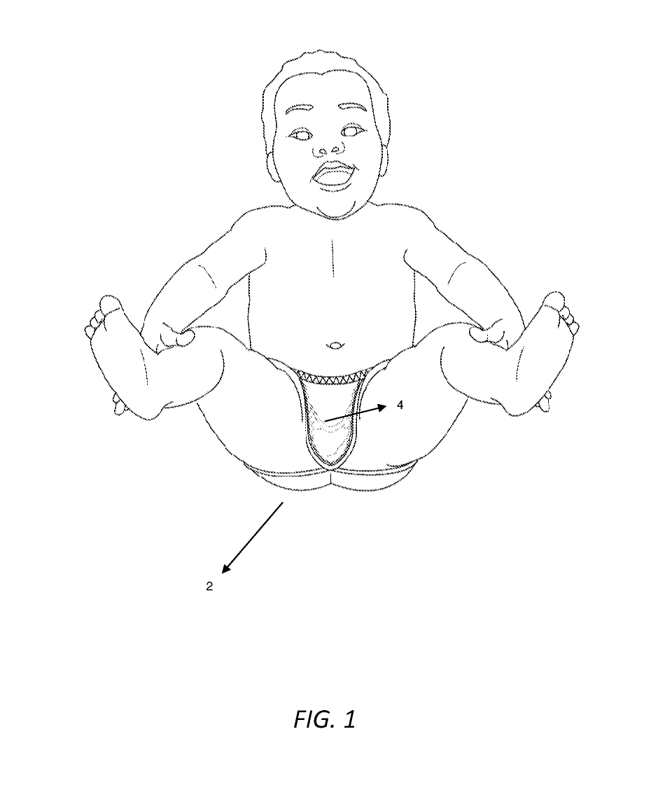

Embodiments of the genital protective cover are illustrated in FIGS. 1-12. As In FIG. 1, front view of the genital protective cover 2 is illustrated when it is worn by a user without any diaper or garments worn over it. The mask 4 completely covers the genital area to protect it from the fecal matter collected in the diaper, garments or any clothing worn over it.

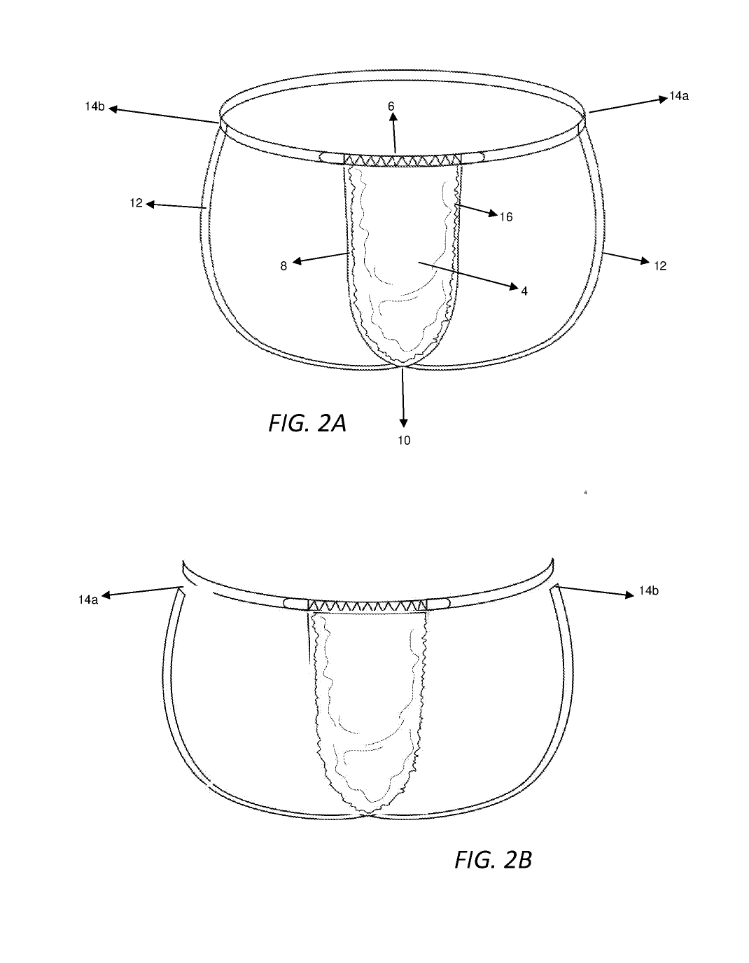

As illustrated in FIGS. 2A-2B, a genital protective cover 2 comprises a mask 4 which is attached to the front part of the waist loop 6 from above. Both sides of the mask are attached to two anterior straps 8 running from straps junction 10 located in perineal region, wherein the two anterior straps 8 are made of the suitable material that, with the help of the two posterior straps 12, keep the mask's edges 16 stick to the area around the genital and tightly enclose the same. The two posterior straps 12 originates from the straps junction 10 and is attachable to the waist loop 6 from left side 14a and right side 14b, wherein the two posterior straps 12 are made of a suitable material and secures the mask 4 tightly from the left and right sides 14a and 14b.

In one embodiment, the two posterior straps 12 may be made of the elastic material and may be attached to the waist loop 6 through Velcro or similar method of fastening.

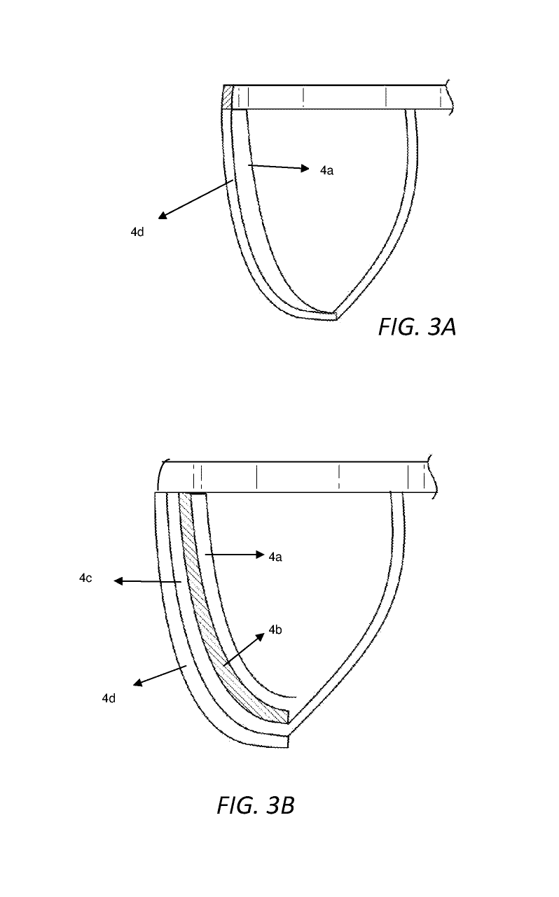

In FIG. 3A, the side view of the multilayered mask 4 is shown with two permeable layers, wherein the interior layer 4a comes into contact with the genital enclosed by the mask 4 and the exterior layer 4d forms outer layer of the mask 4. In one embodiment, the interior layer 4a and exterior layer 4d are made of non-woven material.

In FIG. 3B, the side view of the genital protective cover 2 with four layers of the mask 4 is shown, wherein two additional layers are added namely absorbent pad 4b and a barrier ply 4c are inserted between the interior layer 4a and exterior layer 4d. The absorbent pad 4b absorbs the urine exited from the mask 4. It will be appreciated that the absorbent pad may be made of a hydrophilic material. In another embodiment, a polymer and fibrous material like a wood pulp can be used as an absorbent material. The barrier ply 4c, being made of non-woven material, provides an additional filter in this embodiment.

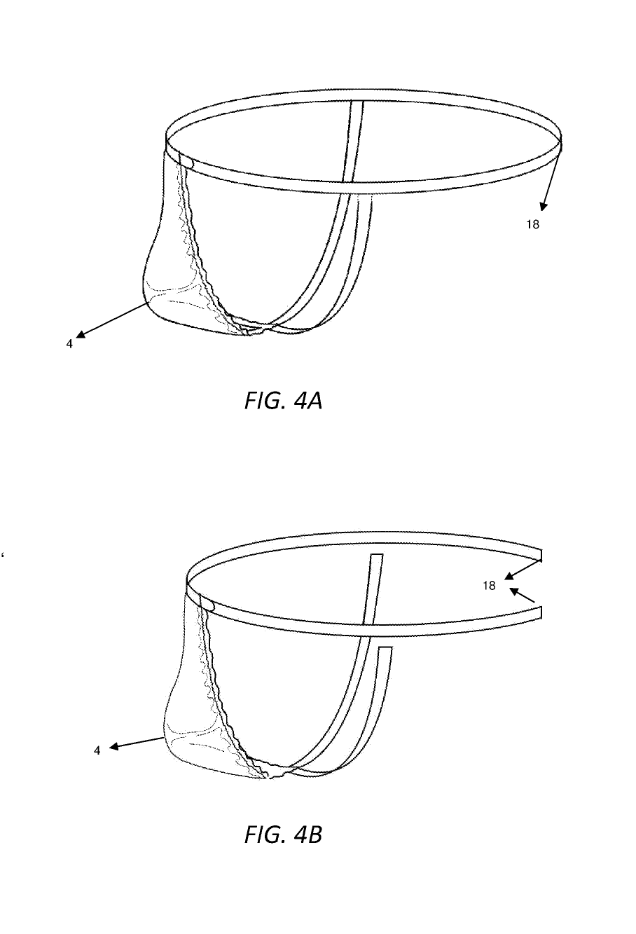

In FIGS. 4A and 4B, the side view of a genital protective cover 2 for male user is shown with an outwardly extended mask 4 forming a bulge-like shape to enclose the male genitalia. In FIG. 4B, the waist loop ends 18 are unfastened.

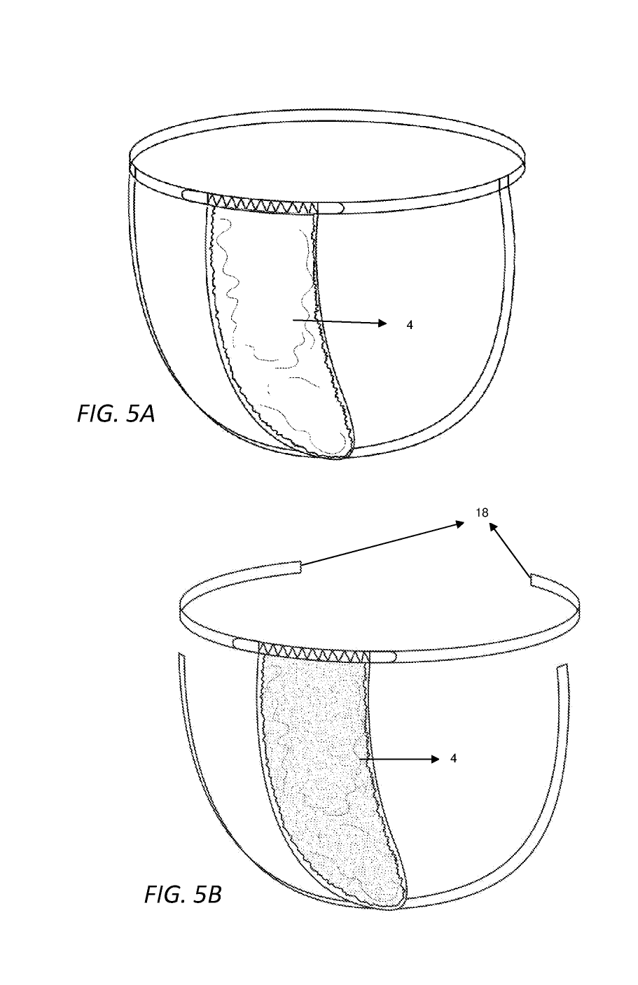

In FIGS. 5A-5B, one embodiment shows the genital protective cover 2 for female users, wherein the mask 4 has a flat shape to fit securely and comfortably over the vaginal region. In FIG. 5B, the unfastened waist loop ends 18 are shown.

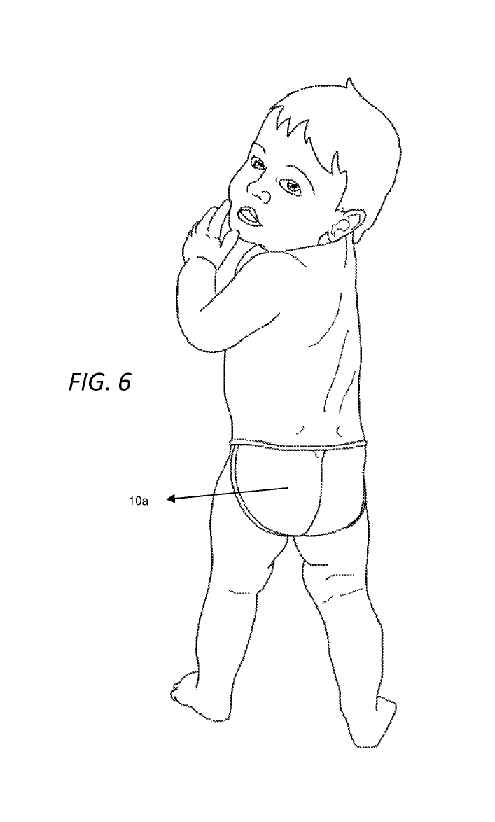

In FIG. 6, a posterior view of the genital protective cover 2 for frontal genital region is illustrated, wherein the wearer's buttocks, anal region 10a, are exposed to diaper or any other garments worn by them so that the fecal matter may be easily collected in such diaper or garments.

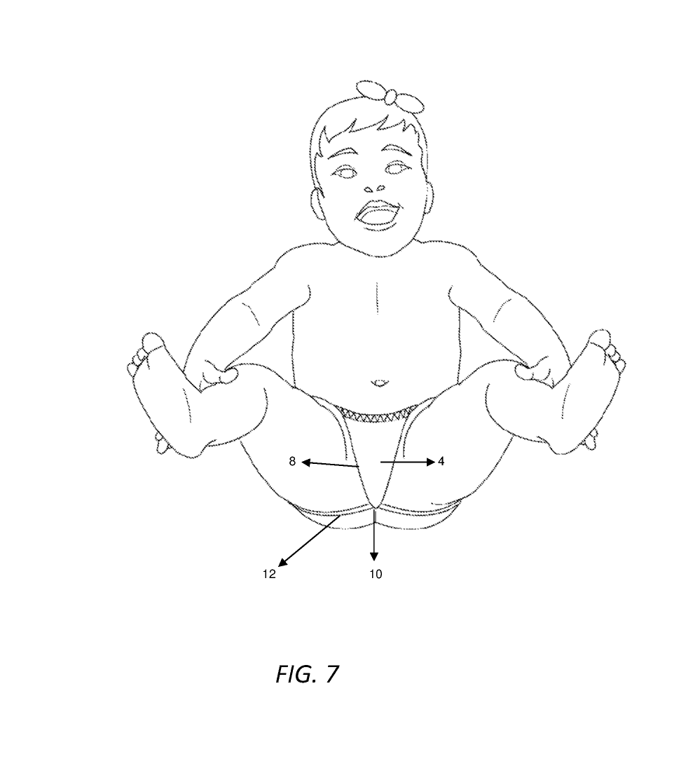

In FIG. 7, one embodiment of the genital protective cover 2 for female wearer is shown, wherein the straps junction 10 and mask 4 are illustrated. The mask 4 for female has flat shape and with the help of the posterior straps 12 and anterior straps 8 covers the vaginal region securely and comfortably.

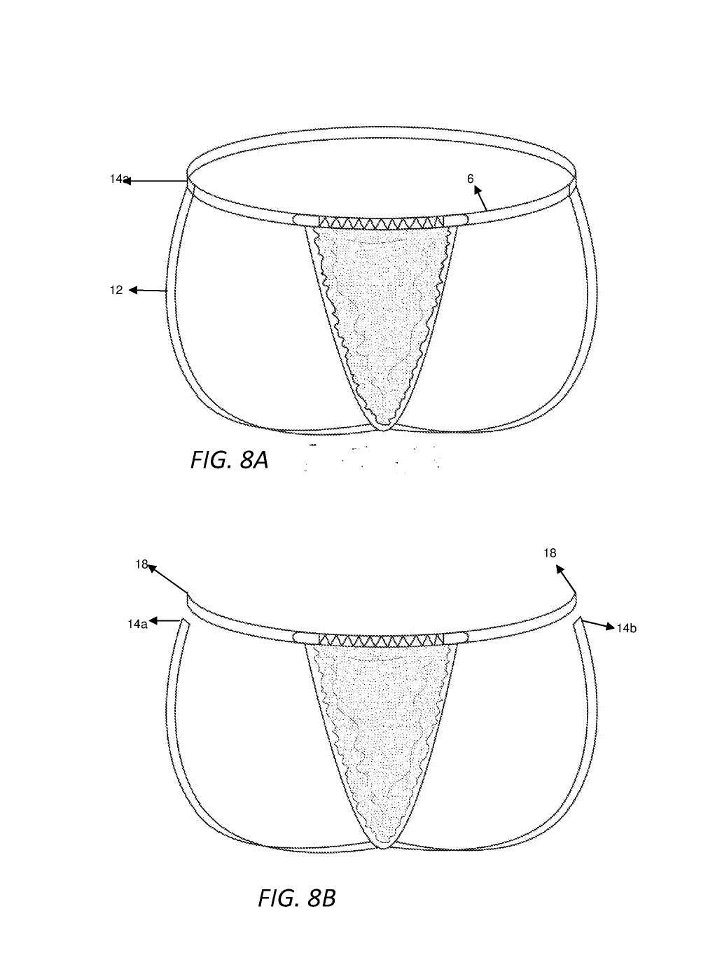

In FIG. 8A, one embodiment is shown with closed posterior straps 12 and waist loop 6, wherein Velcro is used to fasten the waist loop ends 18 and posterior straps ends 14a and 14b. In one embodiment, the waist loop 6 is made of a malleable aluminum wire to give the genital protective cover shape at the proximal end where it is in contact with the waist, distal to the stomach area.

In FIG. 8B, one embodiment with unfastened posterior straps ends 14a and 14b and waist loop ends 18 is shown.

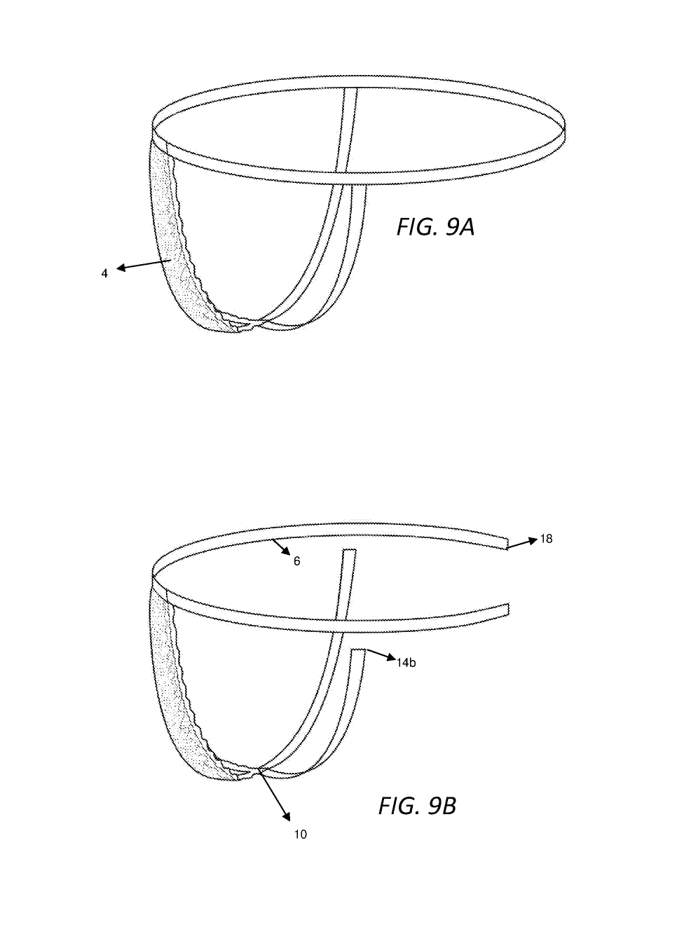

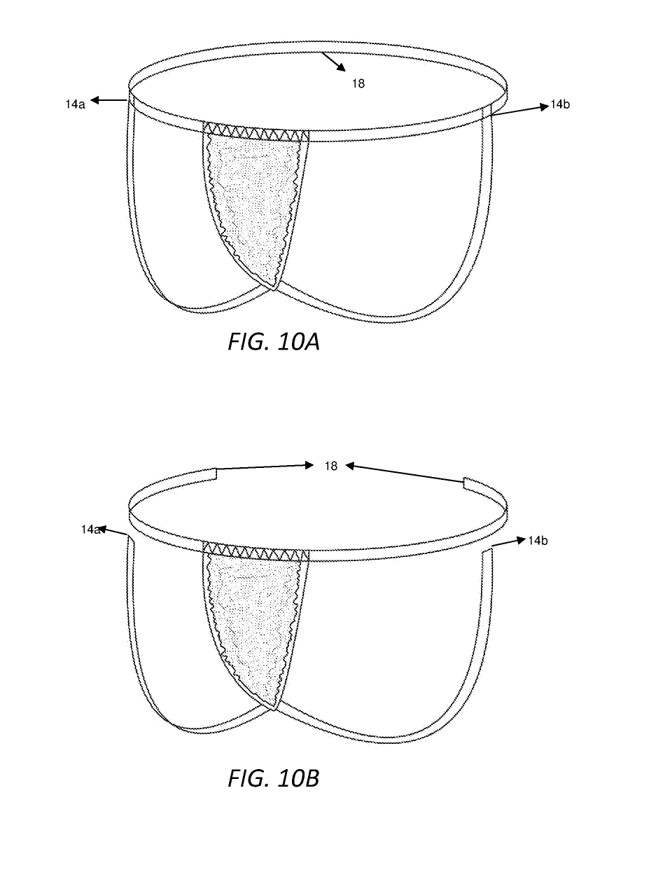

In FIGS. 9A-9B and 10A-10B, different views of the female genital protective cover are shown, wherein fastened and unfastened posterior straps 12 and waist loop 6 through the posterior straps ends 14a and 14b and waist loop ends 18 are illustrated. The ends may be attached with the help of Velcro or any other adequate means of fastening. It will be appreciated that the straps junction located 10 is made of a soft material so that no hurt or annoyance may cause to the user.

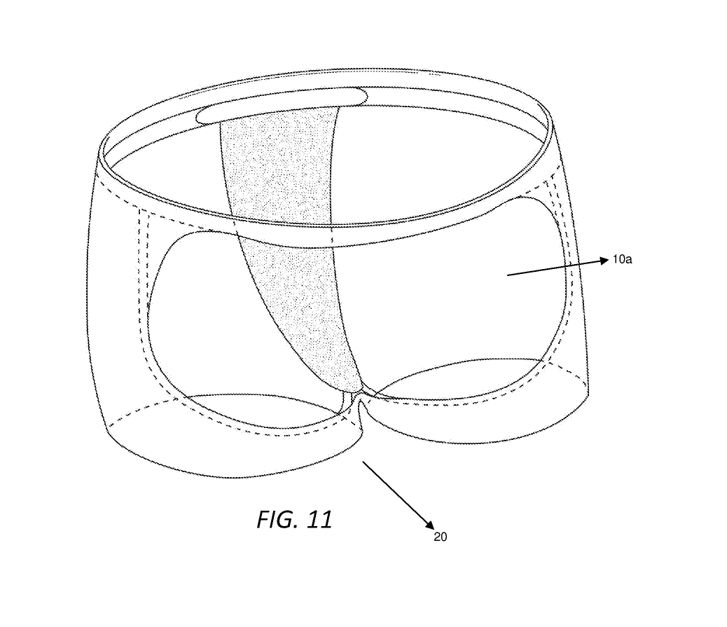

In FIG. 11, one embodiment of a posterior view of the genital protective cover 2 is illustrated. It will be appreciated that the genital protective cover may be used in various configurations in the suitable manner. In one embodiment, the principle of the invention is applied to a shorts-like garment 20 with the posterior side absent exposing the wearer's buttocks and allowing the fecal matter to be collected in the diaper or clothing worn over the shorts-like garment 20. Further in one embodiment, the edges of the short-like garment 20 are made of a stretchable fabric which may make the edges tightly adherent to the sides of the buttocks to prevent the seepage of the fecal matter collected in the diaper or any other garments. Further, in another embodiment, the short-like garment 20 is made of a stretchable material, similar to that which is used for yoga pants, which may keep the shorts-like garment 20 stick to the genital area, part of thighs and sides of the buttocks leaving the anal region 10a exposed to allow the fecal matter to exit and collect in the diaper or clothing worn over the short-like garment 20.

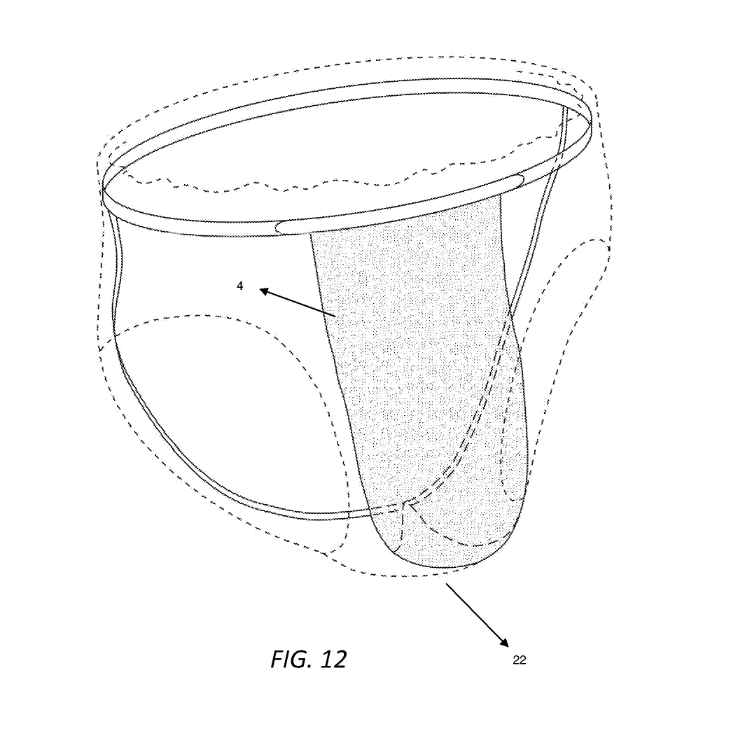

In FIG. 12, the posterior view of the genital protective cover 2 is illustrated wherein the protective cover 2 is used inside a diaper, shorts, incontinence device or any other clothing 22 worn over it.

It will be appreciated that in one embodiment the genital protective cover 2 is manufactured for all age groups ranging from infants to the elderly and infirm people, wherein the protective cover 2 is made in variety of sizes in accordance with the user requirements.

It will be further appreciated that in one embodiment the genital protective cover 2 is made for the patients with medical conditions wherein it is made of the material suitable to protect the wounds.

The foregoing illustrations and embodiments are exemplary only in nature and are not intended to be limiting of the scope of the invention defined by the claims. Further embodiments and examples will be apparent to one of ordinary skill in the art in view of this specification and are within the scope of the claimed invention. Furthermore, the modifications may be made without departing from the spirit and scope of the invention as set forth in the claims hereunder.

What is claimed is:

1. A genital protective cover comprising a suitable fluid penetrable mask locatable over the genital region to prevent exposure of the genital to fecal matter collected in the neighboring buttock region, wherein the genital protective cover is secured by the belt-like waist loop and straps suitably tied to keep the mask tightly stick and fit to the body around the genital region.

2. A genital protective cover as claimed in claim 1 in which the genital protective cover is worn beneath a diaper, undergarments, incontinence device, shorts, training pants, or any clothing.

3. A genital protective cover as claimed in claim 1 in which the edges of the mask are attached from upper side to the front part of the waist loop, from the left and right sides to the anterior straps and from bottom to the posterior straps.

4. A genital protective cover as claimed in claim 1 in which the waist loop encircles the waist.

5. A genital protective cover as claimed in claim 1 in which the two pairs of straps are used to secure the mask from all sides.

6. A genital protective cover as claimed in claim 1 in which two anterior straps, being attached to the left and right sides of the mask, originate from the bottom of the genital protective cover located in the perineal region of wearer and run on both sides of the genitals and join the waist loop from front.

7. A genital protective cover as claimed in claim 1 in which two posterior straps originate from the bottom of the genital protective cover located in the perineal region of the wearer, encircle the sides of the buttocks and joins the waist loop.

8. A genital protective cover as claimed in claim 1 in which the protective cover, waist loop and straps follow the characteristics of wearer with reference to the wearer's age, sex, body size and health condition.

9. A shorts-like genital protective cover comprising a suitable fluid penetrable protective garment locatable over the genital region beneath the undergarments, incontinence product, diaper or any clothing to prevent exposure of wearer's genital to fecal matter collected in the neighboring buttock region, wherein the buttock area is exposed to allow fecal matter to collect in the clothing or diaper worn over it.

10. A shorts-like genital protective cover as claimed in 9 in which the short-like genital protective cover is tightly stick and fit to the body over which it is worn to prevent the seepage of the fecal matter collected in the buttock area.
